(19) United States
(12) Patent Application Publication
(10) Pub. No.: US 2025/0099661 A1
(43) Pub. Date: Mar. 27, 2025

(54) OXYGENATOR AND EXTRACORPOREAL MEMBRANE OXYGENATION DEVICE

(71) Applicants: BEIJING AEROSPACE CHANGFENG CO., LTD., Beijing (CN); BEIHANG UNIVERSITY, Beijing (CN)

(72) Inventors: Zihua SU, Beijing (CN); Mingzhou XU, Beijing (CN); Minghao YUE, Beijing (CN); Jinian LI, Beijing (CN); Yawei WANG, Beijing (CN); Shihang LIN, Beijing (CN); Yubo FAN, Beijing (CN); Zengsheng CHEN, Beijing (CN); Huichao LIU, Beijing (CN); Shiyao ZHANG, Beijing (CN); Yake CHENG, Beijing (CN); Jinyu LI, Beijing (CN); Wenjie YU, Beijing (CN)

(21) Appl. No.: 18/471,673

(22) Filed: Sep. 21, 2023

(51) Int. Cl. A61M 1/16

(52) U.S. Cl. CPC A61M 1/1698; A61M 1/1621

(57) ABSTRACT

Disclosed are an oxygenator and an extracorporeal membrane oxygenation device. The oxygenator includes a housing; an oxygenation chamber, arranged in the housing, and having a blood flow pipeline extend through a blood inlet and a blood outlet; a partition plate, arranged between the housing and the oxygenation chamber, the partition plate is arranged in a same direction as the upper end cover and divide the interior of the housing into a heat medium chamber and a gas chamber. The oxygenator combines the design of a heat medium chamber and a gas chamber to perform brand-new optimization design on a blood flow path, a gas pipeline and a heat medium pipeline of a membrane lung, so as to obtain the best hemodynamic performance, uniform distribution of internal flow fields and pressure fields, small flow retention zone, low blood flow resistance and high gas blood exchange efficiency and heat exchange efficiency.

### TECHNICAL FIELD

The present disclosure belongs to the technical field of medical instrument, and in particular to an oxygenator, and an extracorporeal membrane oxygenation device.

### BACKGROUND

Extracorporeal membrane oxygenation (ECMO), belonging to medical instrument with high technology for critical care, represents an advanced technology in the field of extracorporeal circulation equipment, which involves many disciplines such as biomechanics, fluid mechanics, mechanical engineering, biomaterials and medical science in research and development and is a typical medical-industrial fusion and multidisciplinary crossover product with great research and development difficulty. The development and production capacity of ECMO represents the scientific and technological level of medical devices with high technology in a country to some extent. ECMO plays a vital role in the treatment of critically ill patients with cardiopulmonary diseases caused by infectious diseases or other reasons. Meanwhile, ECMO is also widely used in first aid and treatment of neonatal cardiac or respiratory failure, adult acute respiratory symptom, cardiac arrest, cardiopulmonary bypass assistance during surgery, cardiogenic or postoperative shock, and transport of critically ill patients. At present, there is no domestic ECMO in China, and all the clinical products are imported products, which are scarce in quantity and expensive in price, leading to heavy medical and health burden and constrained public safety emergency response.

Membrane oxygenator (membrane lung) is the key core device in ECMO system, with the main function of blood oxygen exchange and carbon dioxide removal. At present, the manufacturers with membrane lung research and production capacity in the world are concentrated in Germany, the United States, Italy and Japan, such as Maquet of Germany, Medtronic of the United States, Sorin of Italy, Terumo of Japan, Medos of Germany, etc. Among them, Maquet, Medtronic and Sorin occupy the top three in the global market.

The internal main structure of the membrane lung is hollow fiber membrane fiber, which is the key core consumable for membrane lung for gas blood exchange. During the operation of the membrane lung, the chamber inside the hollow membrane fiber is filled with high concentration oxygen, and venous blood flows through the outside of the hollow membrane fiber. The oxygen concentration inside the membrane fiber is higher than that in venous blood outside the membrane fiber, while the carbon dioxide concentration in venous blood is higher than that inside the membrane fiber, so oxygen is transported from the membrane fiber to the blood for blood oxygen exchange, while the carbon dioxide is transported from the blood to the membrane fiber to be removed, thus realizing the gas blood exchange.

In the research and development design of membrane lung, the design of the blood flow path, gas path and heat exchange water path (i.e., heat medium pipeline) is very important, which directly affects the gas blood exchange performance, blood compatibility and heat transfer performance of the membrane lung. For example, if the blood flow path is not well designed, the greater resistance of blood passing through the membrane lung may increase the damage to the membrane fiber when the blood flows through the membrane fiber; and many flow dead zones are generated in the membrane lung, leading to the deposition of harmful substances in blood and the increase of the probability of thrombosis. If gas path of the membrane lung (i.e., gas pipeline) is not well designed, low efficiency of gas blood exchange may be caused, which directly affects the membrane lung function. At present, although ECMO used in clinic practice has treated many patients, there are still problems, such as high incidence of thrombus and low gas exchange efficiency after used for a long time. This is related to the reasons that a large number of flow dead zones caused by poor design of the whole blood flow path and gas path of the membrane lung is prone to blood retention, and the other red blood cells are unable to perform blood oxygen exchange as the red blood cells subjected to blood oxygen exchange cannot be removed in time. Thrombosis directly affects the membrane lung function and reduce the efficiency of gas blood exchange.

### SUMMARY

### (1) Objective

An objective of the present disclosure is to provide an oxygenator and an extracorporeal membrane oxygenation device, so as to solve the technical problem of blood retention caused by the existence of flow dead zones in the oxygenator in the prior art.

### (2) Technical Solution

In order to solve the above problems, in a first aspect of the present disclosure, an oxygenator is provided. The oxygenator includes a housing, provided with an upper end cover and a lower end cover arranged opposite to each other, the upper end cover is connected to the lower end cover via a side wall, a blood inlet is arranged at a center of the upper end cover, and a blood outlet is arranged at one end of the lower end cover of the housing close to the side wall; an oxygenation chamber, arranged in the housing, blood is fed into the oxygenation chamber through the blood inlet, and the blood oxygenated is discharged through the blood outlet; and a partition plate, arranged between the housing and the oxygenation chamber, the partition plate is arranged in a same direction as the upper end cover, and divides an interior of the housing into a heat medium chamber and a gas chamber.

Furthermore, the side wall of the housing is provided with a heat medium inlet and a heat medium outlet. A heat medium is fed into the heat medium chamber through the heat medium inlet, and the heat medium subjected to heat exchange is discharged through the heat medium outlet.

Furthermore, an interior of the heat medium chamber is further provided with a first isolation part and a second isolation part. The first isolation part and the second isolation part separate the heat medium chamber into a first heat medium chamber and a second heat medium chamber. The first heat medium chamber is communicated with the heat medium inlet, and the second heat medium chamber is communicated with the heat medium outlet. The first heat medium chamber is communicated with the second heat medium chamber via a heat medium pipeline, and the heat medium pipeline extends through the oxygenation chamber.

Furthermore, the first isolation part is arranged between the housing and the oxygenation chamber and at a side close to the blood outlet. The second isolation part is arranged between the housing and the oxygenation chamber and at a side away from the blood outlet. The first isolation part and the second isolation part divide the heat medium chamber into the first heat medium chamber and the second heat medium chamber having a same size.

Furthermore, the heat medium inlet and the heat medium outlet are arranged on one end of the side wall of the housing close to the blood outlet. The oxygenation chamber is of a quadrangular prism structure, and two opposite side surfaces of the oxygenation chamber are connected via the heat medium pipeline.

Furthermore, the side wall of the housing is provided with a gas inlet and a gas outlet. An oxygen-containing gas is fed into the gas chamber through the gas inlet, and the gas subjected to gas blood exchange is exhausted through the gas outlet.

Furthermore, an interior of the gas chamber is provided with a third isolation part and a fourth isolation part. The third isolation part and the fourth isolation part separate the gas chamber into a first gas chamber and a second gas chamber. The first gas chamber is communicated with the gas inlet, and the second gas chamber is communicated with the gas outlet. The first gas chamber is communicated with the second gas chamber via a gas pipeline, and the gas pipeline extends through the oxygenation chamber.

Furthermore, the third isolation part and the fourth isolation part are respectively arranged on both ends of the gas chamber. The third isolation part and the fourth isolation part divide the gas chamber into the first gas chamber and the second gas chamber having a same size.

Furthermore, the gas inlet is arranged on one end of the side wall of the housing away from the blood outlet. The gas outlet is arranged on one end of the side wall of the housing close to the blood outlet. The oxygenation chamber is of a quadrangular prism structure, and two opposite side surfaces of the oxygenation chamber are connected via the gas pipeline.

According to another aspect of the present disclosure, an extracorporeal membrane oxygenation device is provided, including the oxygenator of any one of above technical solutions.

### (3) Beneficial Effects

Above technical solutions of the present disclosure have the following beneficial effects:

On the basis of multi-objective and multi-parameter optimization research on hemodynamics, gas blood exchange and the like, the oxygenator combines the design of the heat medium chamber and the gas chamber to perform brand-new optimization design on a blood flow path, a gas pipeline and a heat medium pipeline of a membrane lung, so as to obtain the best hemodynamic performance, uniform distribution of internal flow fields and pressure fields, small flow retention zone, low blood flow resistance and high gas blood exchange efficiency and heat exchange efficiency. Therefore, the efficacy of the oxygenator used for a long time is improved, the probability of thrombosis caused by the oxygenator used for a long time is reduced, and the blood compatibility of the oxygenator is improved.

### BRIEF DESCRIPTION OF THE DRAWINGS

FIG. 1 is a structural schematic diagram of an oxygenator in accordance with an embodiment of the present disclosure;

FIG. 2 is a structural schematic diagram of an oxygenator in accordance with another embodiment of the present disclosure;

FIG. 3 is a structural schematic diagram of an oxygenator in accordance with yet another embodiment of the present disclosure;

FIG. 4 is a perspective view of an oxygenator in accordance with an embodiment of the present disclosure;

FIG. 5 is a perspective view of an oxygenator in accordance with another embodiment of the present disclosure;

FIG. 6 is a perspective view of an oxygenator in accordance with yet another embodiment of the present disclosure;

FIG. 7 is a structural schematic diagram of an oxygenation chamber in accordance with an embodiment of the present disclosure;

FIG. 8 is a structural schematic diagram of a heat medium chamber in accordance with an embodiment of the present disclosure;

FIG. 9 is a structural schematic diagram of a gas chamber in accordance with an embodiment of the present disclosure.

### REFERENCE NUMERALS

100—housing; 110—blood inlet; 120—blood outlet; 130—heat medium inlet; 140—heat medium outlet; 150—gas inlet; 160—gas outlet; 170—first exhaust port; 180—second exhaust port; 200—oxygenation chamber; 300—partition plate; 400—heat medium chamber; 410—first isolation part; 420—second isolation part; 430—first heat medium chamber; 440—second heat medium chamber; 500—gas chamber; 510—third isolation part; 520—fourth isolation part; 530—first gas chamber; 540—second gas chamber; 600—upper end cover; 700—lower end cover.

### DETAILED DESCRIPTION OF THE EMBODIMENTS

To make the above objectives, features and advantages of the present disclosure more clearly and understandably, the present disclosure is further described in detail below in conjunction with specific embodiments and with reference to the accompanying drawings. It should be understood that these descriptions are exemplary only and are not intended to limit the scope of the present disclosure. Further, in the following description, a description of well-known structures and techniques is omitted to avoid unnecessarily confusing the concepts of the present disclosure.

A schematic diagram of a layer structure according to an embodiment of the present disclosure is shown in the drawings. These drawings are not drawn to scale, in which some details are enlarged or omitted for clarity. The shapes of various regions and layers shown in the drawings, as well as their relative sizes and positional relationships, are only exemplary, and may actually be deviated due to manufacturing tolerances or technical limitations. Moreover, those skilled in the art can additionally design regions/layers with different shapes, sizes and relative positions according to actual needs.

Apparently, the described embodiments are a part rather than all embodiments. All other embodiments obtained by a person of ordinary skill in the art based on the embodiments of the present disclosure without creative efforts shall fall within the scope of the present disclosure.

In addition, the technical features involved in the different embodiments of the present disclosure described below may be combined with each other as long as they do not constitute a conflict with each other.

The present disclosure will be described in more detail below with reference to the accompanying drawings. Throughout the various drawings, like elements are denoted by like reference numerals. For the sake of clarity, various parts in the drawings are not drawn to scale.

At present, the oxygenator in the prior art used for a long time has some problems in clinical use, such as high incidence of thrombosis, decreased efficiency of gas blood exchange, increased resistance of blood flowing through a membrane lung, etc. These problems will reduce the blood compatibility of the membrane lung, affect the efficacy and function of the membrane lung and increase the risk of patients.

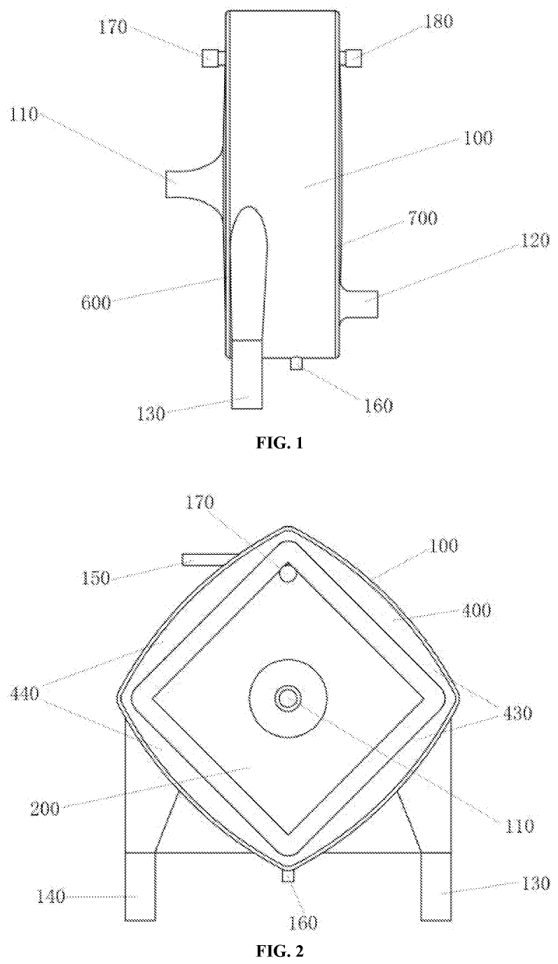

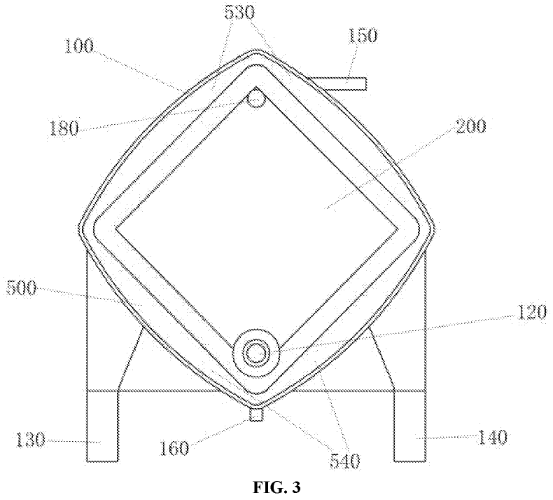

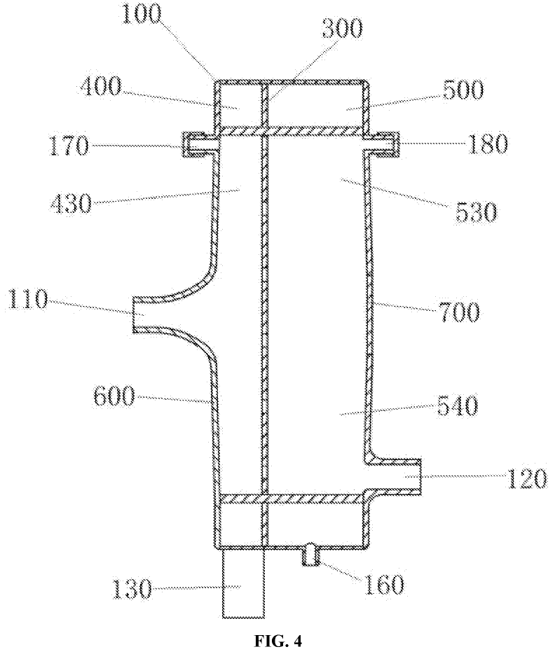

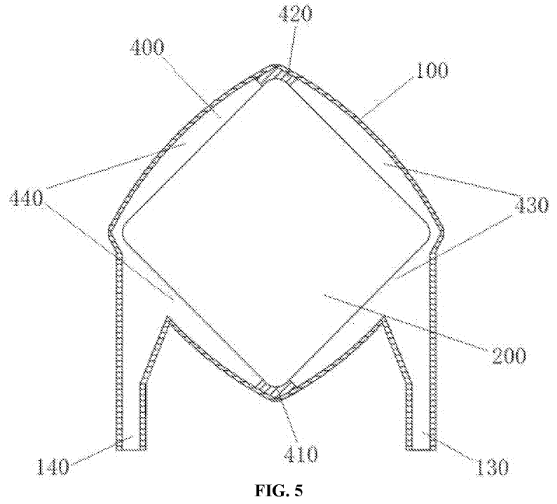

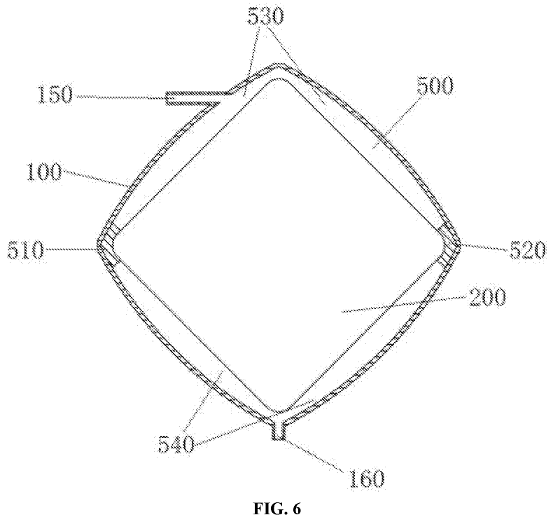

As shown in FIGS. 1-6, in an embodiment of the present disclosure, an oxygenator is provided. The oxygenator may include a housing 100, an oxygenation chamber 200, and a partition plate 300. The housing 100 is provided with an upper end cover 600 and a lower end cover 700 arranged opposite to each other, the upper end cover 600 and the lower end cover 700 are connected through a side wall, a blood inlet 110 is arranged at the center of the upper end cover 600, and a blood outlet 120 is arranged at one end of the lower end cover 700 of the housing 100 close to the side wall. The oxygenation chamber 200 is arranged in the housing 100, blood is fed into the oxygenation chamber 200 through the blood inlet 110, and the blood then is oxygenated and discharged through the blood outlet 120. The partition plate 300 is arranged between the housing 100 and the oxygenation chamber 200, the partition plate 300 is arranged in a same direction as the upper end cover 600, and divides the interior of the housing 100 into a heat medium chamber 400 and a gas chamber 500.

On the basis of multi-objective and multi-parameter optimization research on hemodynamics, gas blood exchange and the like, the oxygenator combines the design of the heat medium chamber 400 and the gas chamber 500 to perform brand-new optimization design on a blood flow path (i.e., the blood flows into the oxygenation chamber 200 through the blood inlet 110, and then flows out through the blood outlet 120 after subjected to heat exchange with the heat medium and gas blood exchange with the gas), a gas pipeline and a heat medium pipeline of the membrane lung, so as to obtain the best hemodynamic performance, uniform distribution of internal flow fields and pressure fields, small flow retention zone, low blood flow resistance and high gas blood exchange efficiency and heat exchange efficiency. Therefore, the efficacy of the oxygenator used for a long time is improved, the probability of thrombosis caused by the oxygenator used for a long time is reduced, and the blood compatibility of the oxygenator is improved.

In an alternative embodiment, the partition plate 300 extends through the oxygenation chamber 200. The partition plate 300 in the oxygenation chamber 200 is provided with multiple through holes. The blood flows into the oxygenation chamber 200 through the blood inlet 110, flows through the heat medium pipeline of the heat medium chamber 400 for heat exchange, passes through the through holes, then flows through the gas pipeline of the gas chamber 500 for gas exchange to complete the respiratory process, and finally flows out through the blood outlet 120.

In an alternative embodiment, the housing 100 may further be provided with a first exhaust port 170. The first exhaust port 170 is provided on the upper end cover 600 and communicated with the heat medium chamber 400. The first exhaust port 170 is configured for injecting a heat medium into the heat medium chamber 400 to exhaust the air from the heat medium chamber 400 before the oxygenator is used.

In an alternative embodiment, one end of the first exhaust port 170 away from the heat medium chamber 400 is provided with a sealing cover.

In an alternative embodiment, the housing 100 may further be provided with a second exhaust port 180. The second exhaust port 180 is provided on the lower end cover 700, and communicated with the oxygenation chamber 200. The second exhaust port 180 is configured for injecting blood into the oxygenation chamber 200 to exhaust the air from the oxygenation chamber 200 before the oxygenator is used.

In an alternative embodiment, the second exhaust port 180 is also configured to exhaust a small amount of gas generated in the oxygenation chamber 200 during the use of the oxygenator.

In an alternative embodiment, one end of the second exhaust port 180 away from the heat medium chamber 200 is provided with a sealing cover.

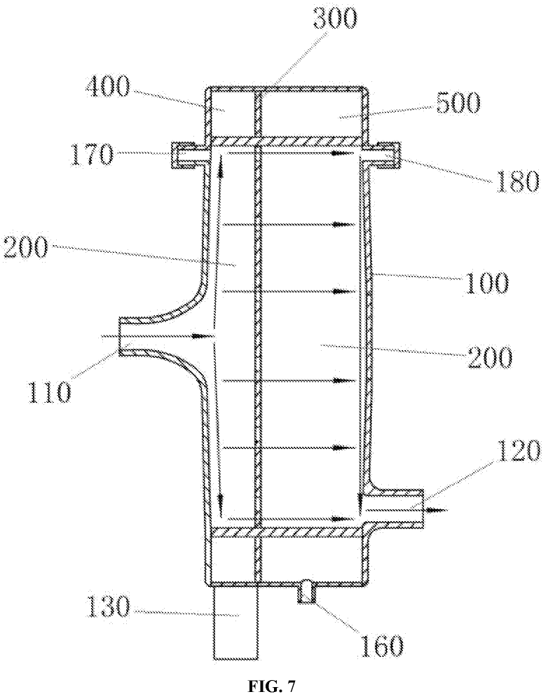

FIG. 7 is a structural schematic diagram of an oxygenator in accordance with an embodiment of the present disclosure.

As shown in FIG. 7, the direction of each arrow in FIG. 7 is a path of the blood flow (blood). The blood in the blood pipeline flows into the oxygenation chamber 200 through the blood inlet 110, flows through the heat medium pipeline of the heat medium chamber 400 for heat exchange, passes through the through holes, then flows through the gas pipeline of the gas chamber 500 for gas exchange to complete the respiratory process, and finally flows out through the blood outlet 120.

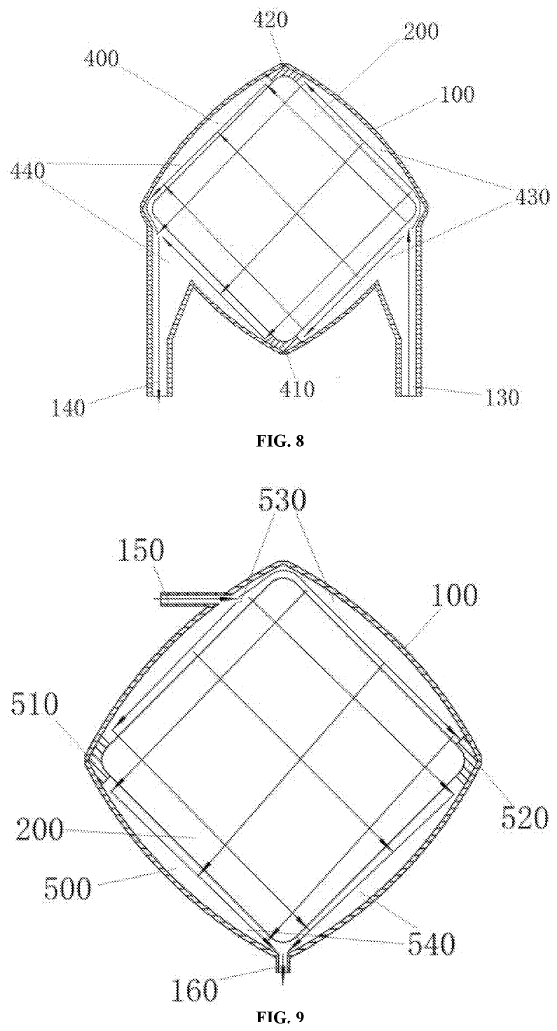

FIG. 8 is a structural schematic diagram of a heat medium chamber in accordance with an embodiment of the present disclosure.

As shown in FIG. 8, in an alternative embodiment, the side wall of the housing 100 is provide with a heat medium inlet 130 through which the heat medium is conveyed into the heat medium chamber 400.

In an alternative embodiment, the side wall of the housing 100 is provided with a heat medium outlet 140 through which the heat medium subjected to heat exchange in the heat medium chamber 400 is exhausted.

In an alternative embodiment, the interior of the heat medium chamber 400 is further provided with a first isolation part 410 and a second isolation part 420. The first isolation part 410 and the second isolation part 420 separate the heat medium chamber 400 into a first heat medium chamber 430 and a second heat medium chamber 440.

In an alternative embodiment, the first heat medium chamber 430 is communicated with the heat medium inlet 130, and the second heat medium chamber 440 is communicated with the heat medium outlet 140.

In an alternative embodiment, the first heat medium chamber 430 is communicated with the second heat medium chamber 440 via the heat medium pipeline, and the heat medium pipeline extends through the oxygenation chamber 200.

In an alternative embodiment, the first isolation part 410 is arranged between the housing 100 and the oxygenation chamber 200 and at a side close to the blood outlet 120.

In an alternative embodiment, the second isolation part 420 is arranged between the housing 100 and the oxygenation chamber 200 and at a side away from the blood outlet 120.

In an alternative embodiment, the first isolation part 410 and the second isolation part 420 divide the heat medium chamber 400 into the first heat medium chamber 430 and the second heat medium chamber 440 having the same size.

In an alternative embodiment, the heat medium inlet 130 and the heat medium outlet 140 are arranged on one end of the side wall of the housing 100 close to the blood outlet 120.

In an alternative embodiment, the oxygenation chamber 200 is of a quadrangular prism structure, and two opposite side surfaces of the oxygenation chamber 200 are connected via the heat medium pipeline.

As shown in FIG. 8, the direction of each arrow in FIG. 8 is a path of the heat medium flow, the heat medium flows into the first heat medium chamber 430 through the heat medium inlet 130, the heat medium in the first heat medium chamber 430 flows into the second heat medium chamber 440 via the heat medium pipeline which extends through the oxygenation chamber 200, and the heat medium in the heat medium pipeline exchanges heat with the blood flow in the oxygenation chamber 200, and the heat medium subjected to heat exchange in the second heat medium chamber 440 flows out through the heat medium outlet 140.

FIG. 9 is a structural schematic diagram of a gas chamber in accordance with an embodiment of the present disclosure.

As shown in FIG. 9, in an alternative embodiment, the side wall of the housing 100 is provided with a gas inlet 150 through which an oxygen-containing gas is conveyed into the gas chamber 500.

In an alternative embodiment, the side wall of the housing 100 is provided with a gas outlet 160 through which the oxygen-containing gas reacted in the gas chamber 500 is exhausted.

In an alternative embodiment, the interior of the gas chamber 500 is provided with a third isolation part 510 and a fourth isolation part 520. The third isolation part 510 and the fourth isolation part 520 separate the gas chamber 500 into a first gas chamber 530 and a second gas chamber 540.

In an alternative embodiment, the first gas chamber 530 is communicated with the gas inlet 150, and the second gas chamber 540 is communicated with the gas outlet 160.

In an alternative embodiment, the first gas chamber 530 is communicated with the second gas chamber 540 via a gas pipeline, and the gas pipeline extends through the oxygenation chamber 200.

In an alternative embodiment, the third isolation part 510 and the fourth isolation part 520 are respectively arranged at both ends of the blood outlet 120.

In an alternative embodiment, the third isolation part 510 and the fourth isolation part 520 divide the gas chamber 500 into the first gas chamber 530 and a second gas chamber 540 having the same size.

In an alternative embodiment, the gas inlet 150 is arranged on one end of the side wall of the housing 100 far away from the blood outlet 120.

In an alternative embodiment, the gas outlet 160 is arranged on one end of the side wall of the housing 100 close to the blood outlet 120.

In an alternative embodiment, the oxygenation chamber 200 is of a quadrangular prism structure, and two opposite side surfaces of the oxygenation chamber 200 are connected via the gas pipeline.

As shown in FIG. 9, the direction of each arrow in FIG. 9 is a path of the gas flow. The gas is fed into the first gas chamber 530 through the gas inlet 150, the gas in the first gas chamber 530 enters into the second gas chamber 540 via the gas pipeline which extends through the oxygenation chamber 200, the gas in the gas pipeline exchanges gas with the blood flow in the oxygenation chamber 200, and the gas subjected to gas exchange in the second gas chamber 540 is exhausted through the gas outlet 160.

In another embodiment of the present disclosure, an extracorporeal membrane oxygenation device is provided, which may include the oxygenator of any one of the above technical solutions.

The present disclosure is intended to protect an oxygenator and an extracorporeal membrane oxygenation device. The oxygenator may include a housing 100; an oxygenation chamber 200, and a partition plate 300. The housing 100 is provided with an upper end cover 600 and a lower end cover 700 arranged opposite to each other, the upper end cover 600 and the lower end cover 700 are connected through a side wall, a blood inlet 110 is arranged at the center of the upper end cover 600, and a blood outlet 120 is arranged at one end of the lower end cover 700 of the housing 100 close to the side wall. The oxygenation chamber 200 is arranged in the housing 100, blood is fed into the oxygenation chamber 200 through the blood inlet 110, and the blood oxygenated is discharged through the blood outlet 120. The partition plate 300 is arranged between the housing 100 and the oxygenation chamber 200, the partition plate 300 is arranged in a same direction as the upper end cover 600, and divides the interior of the housing 100 into a heat medium chamber 400 and a gas chamber 500. On the basis of multi-objective and multi-parameter optimization research on hemodynamics, gas blood exchange and the like, the oxygenator combines the design of the heat medium chamber 400 and the gas chamber 500 to perform brand-new optimization design on a blood flow path (i.e., the blood flows into the oxygenation chamber 200 through the blood inlet 110, and then flows out through the blood outlet 120 after subjected to heat exchange with the heat medium and gas blood exchange with the gas), a gas pipeline and a heat medium pipeline of the membrane lung, so as to obtain the best hemodynamic performance, uniform distribution of internal flow fields and pressure fields, small flow retention zone, low blood flow resistance and high gas blood exchange efficiency and heat exchange efficiency. Therefore, the efficacy of the oxygenator used for a long time is improved, the probability of thrombosis caused by the oxygenator used for a long time is reduced, and the blood compatibility of the oxygenator is improved.

It should be understood that the above specific embodiments of the present disclosure are only used to illustrate or explain the principles of the present disclosure, and do not constitute limitations of the present disclosure. Any modification, equivalent replacement, improvement, etc. made without departing from the spirit and principles of the present disclosure should be included within the scope of the present disclosure. In addition, the appended claims of the present disclosure are intended to cover all variations and modifications falling within the scope and boundaries of the appended claims or equivalent forms of such scope and boundaries.

What is claimed is:

1. An oxygenator, comprising:
a housing (100), provided with an upper end cover (600) and a lower end cover (700) arranged opposite to each other, wherein the upper end cover (600) is connected to the lower end cover (700) via a side wall, a blood inlet (110) is arranged at a center of the upper end cover (600), and a blood outlet (120) is arranged at one end of the lower end cover (700) of the housing (100) close to the side wall;
an oxygenation chamber (200), arranged in the housing (100), wherein blood is fed into the oxygenation chamber (200) through the blood inlet (110), and the blood oxygenated is discharged through the blood outlet (120); and
a partition plate (300), arranged between the housing (100) and the oxygenation chamber (200), wherein the partition plate (300) is arranged in a same direction as the upper end cover (600), and divides an interior of the housing (100) into a heat medium chamber (400) and a gas chamber (500).

2. The oxygenator according to claim 1, wherein
the side wall of the housing (100) is provided with a heat medium inlet (130) and a heat medium outlet (140), a heat medium is fed into the heat medium chamber (400) through the heat medium inlet (130), and the heat medium subjected to heat exchange is discharged through the heat medium outlet (140).

3. The oxygenator according to claim 2, wherein
an interior of the heat medium chamber (400) is further provided with a first isolation part (410) and a second isolation part (420), and the first isolation part (410) and the second isolation part (420) separate the heat medium chamber (400) into a first heat medium chamber (430) and a second heat medium chamber (440);
the first heat medium chamber (430) is communicated with the heat medium inlet (130), and the second heat medium chamber (440) is communicated with the heat medium outlet (140);
the first heat medium chamber (430) is communicated with the second heat medium chamber (440) via a heat medium pipeline, and the heat medium pipeline extends through the oxygenation chamber (200).

4. The oxygenator according to claim 3, wherein
the first isolation part (410) is arranged between the housing (100) and the oxygenation chamber (200) and at a side close to the blood outlet (120);
the second isolation part (420) is arranged between the housing (100) and the oxygenation chamber (200) and at a side away from the blood outlet (120);
the first isolation part (410) and the second isolation part (420) divide the heat medium chamber (400) into the first heat medium chamber (430) and a second heat medium chamber (440) having a same size.

5. The oxygenator according to claim 4, wherein
the heat medium inlet (130) and the heat medium outlet (140) are arranged on one end of the side wall of the housing (100) close to the blood outlet (120);
the oxygenation chamber (200) is of a quadrangular prism structure, and two opposite side surfaces of the oxygenation chamber (200) are connected via the heat medium pipeline.

6. The oxygenator according to claim 1, wherein
the side wall of the housing (100) is provided with a gas inlet (150) and a gas outlet (160), an oxygen-containing gas is fed into the gas chamber (500) through the gas inlet (150), and the gas subjected to gas blood exchange is exhausted through the gas outlet (160).

7. The oxygenator according to claim 6, wherein
an interior of the gas chamber (500) is provided with a third isolation part (510) and a fourth isolation part (520), and the third isolation part (510) and the fourth isolation part (520) separate the gas chamber (500) into a first gas chamber (530) and a second gas chamber (540);
the first gas chamber (530) is communicated with the gas inlet (150), and the second gas chamber (540) is communicated with the gas outlet (160).
the first gas chamber (530) is communicated with the second gas chamber (540) via a gas pipeline, and the gas pipeline extends through the oxygenation chamber (200).

8. The oxygenator according to claim 7, wherein
the third isolation part (510) and the fourth isolation part (520) are respectively arranged at both ends of the gas chamber (500);
the third isolation part (510) and the fourth isolation part (520) divide the gas chamber (500) into the first gas chamber (530) and a second gas chamber (540) having a same size.

9. The oxygenator according to claim 8, wherein
the gas inlet (150) is arranged on one end of the side wall of the housing (100) away from the blood outlet (120);
the gas outlet (160) is arranged on one end, close to the blood outlet (120) of the side wall of the housing (100) close to the blood outlet (120);
the oxygenation chamber (200) is of a quadrangular prism structure, and two opposite side surfaces of the oxygenation chamber (200) are connected via the gas pipeline.

10. The oxygenator according to claim 2, wherein
the side wall of the housing (100) is provided with a gas inlet (150) and a gas outlet (160), an oxygen-containing gas is fed into the gas chamber (500) through the gas inlet (150), and the gas subjected to gas blood exchange is exhausted through the gas outlet (160).

11. The oxygenator according to claim 3, wherein
the side wall of the housing (100) is provided with a gas inlet (150) and a gas outlet (160), an oxygen-containing gas is fed into the gas chamber (500) through the gas inlet (150), and the gas subjected to gas blood exchange is exhausted through the gas outlet (160).

12. The oxygenator according to claim 4, wherein
the side wall of the housing (100) is provided with a gas inlet (150) and a gas outlet (160), an oxygen-containing gas is fed into the gas chamber (500) through the gas inlet (150), and the gas subjected to gas blood exchange is exhausted through the gas outlet (160).

13. The oxygenator according to claim 5, wherein
the side wall of the housing (100) is provided with a gas inlet (150) and a gas outlet (160), an oxygen-containing gas is fed into the gas chamber (500) through the gas inlet (150), and the gas subjected to gas blood exchange is exhausted through the gas outlet (160).

14. An extracorporeal membrane oxygenation device, comprising the oxygenator according to claim 1.

15. The extracorporeal membrane oxygenation device according to claim 14, wherein
the side wall of the housing (100) is provided with a heat medium inlet (130) and a heat medium outlet (140), a heat medium is fed into the heat medium chamber (400) through the heat medium inlet (130), and the heat medium subjected to heat exchange is discharged through the heat medium outlet (140).

16. The extracorporeal membrane oxygenation device according to claim 15, wherein
an interior of the heat medium chamber (400) is further provided with a first isolation part (410) and a second isolation part (420), and the first isolation part (410) and the second isolation part (420) separate the heat medium chamber (400) into a first heat medium chamber (430) and a second heat medium chamber (440);
the first heat medium chamber (430) is communicated with the heat medium inlet (130), and the second heat medium chamber (440) is communicated with the heat medium outlet (140);
the first heat medium chamber (430) is communicated with the second heat medium chamber (440) via a heat medium pipeline, and the heat medium pipeline extends through the oxygenation chamber (200).

17. The extracorporeal membrane oxygenation device according to claim 16, wherein
the first isolation part (410) is arranged between the housing (100) and the oxygenation chamber (200) and at a side close to the blood outlet (120);
the second isolation part (420) is arranged between the housing (100) and the oxygenation chamber (200) and at a side away from the blood outlet (120);
the first isolation part (410) and the second isolation part (420) divide the heat medium chamber (400) into the first heat medium chamber (430) and a second heat medium chamber (440) having a same size.

18. The extracorporeal membrane oxygenation device according to claim 17, wherein
the heat medium inlet (130) and the heat medium outlet (140) are arranged on one end of the side wall of the housing (100) close to the blood outlet (120);
the oxygenation chamber (200) is of a quadrangular prism structure, and two opposite side surfaces of the oxygenation chamber (200) are connected via the heat medium pipeline.

19. The extracorporeal membrane oxygenation device according to claim 14, wherein
the side wall of the housing (100) is provided with a gas inlet (150) and a gas outlet (160), an oxygen-containing gas is fed into the gas chamber (500) through the gas inlet (150), and the gas subjected to gas blood exchange is exhausted through the gas outlet (160).

20. The extracorporeal membrane oxygenation device according to claim 19, wherein
an interior of the gas chamber (500) is provided with a third isolation part (510) and a fourth isolation part (520), and the third isolation part (510) and the fourth isolation part (520) separate the gas chamber (500) into a first gas chamber (530) and a second gas chamber (540);
the first gas chamber (530) is communicated with the gas inlet (150), and the second gas chamber (540) is communicated with the gas outlet (160).
the first gas chamber (530) is communicated with the second gas chamber (540) via a gas pipeline, and the gas pipeline extends through the oxygenation chamber (200).
